(19) United States
(12) Patent Application Publication
(10) Pub. No.: US 2018/0357366 A1
(43) Pub. Date: Dec. 13, 2018

(54) METHOD FOR DETERMINING COPY-NUMBER VARIATION IN SAMPLE COMPRISING MIXTURE OF NUCLEIC ACIDS

(71) Applicant: GREEN CROSS GENOME CORPORATION, Yongin-si, Gyeonggi-do (KR)

(72) Inventors: Eun-Hae CHO, Yongin-si (KR); Junnam LEE, Yongin-si (KR); Young-Joo JEON, Yongin-si (KR); Ja-Hyun JANG, Yongin-si (KR); Taeheon LEE, Yongin-si (KR)

(73) Assignee: GREEN CROSS GENOME CORPORATION, Yongin-si, Gyeonggi-do (KR)

(21) Appl. No.: 15/781,177

(22) PCT Filed: Dec. 4, 2015
(86) PCT No.: PCT/KR2015/013210

§ 371 Date: Jun. 4, 2018

(51) Int. Cl. G06F 19/22; G06F 19/18

(52) U.S. Cl. CPC G06F 19/22; C12Q 1/6883; G06F 19/18

(57) ABSTRACT

A method for determining copy number variation in a mixture of nucleic acids, which are known or believed to be different in terms of the amount of one or more target sequences. The method for determining variation may be used to chromosomal copy number variation which is associated with or believed to be associated with fetal diseases. Chromosomal copy number variations that may be determined according to the method may include trisomy and monosomy of any one or more of chromosomes 1-22, X and Y, polysomy for the full-length nucleic acid sequence, and deletion and/or duplication of any one or more sequence fragments of chromosomes, and thus the method is useful for the analysis of fetal gender and copy number variation.

### TECHNICAL FIELD

The present invention relates to a method for detecting fetal gender and copy number abnormalities, and more particularly to a noninvasive method for detecting fetal chromosomal abnormalities, which comprises extracting DNA from a maternal biological sample, obtaining reads from the DNA, normalizing chromosomal regions, and randomly permuting reference chromosomes.

### BACKGROUND ART

Conventional prenatal tests for fetal chromosomal abnormalities include ultrasonography, blood marker testing, amniocentesis, chorionic villus sampling, percutaneous umbilical cord blood sampling, and the like (Malone F D, et al. 2005; Mujezinovic F, et al. 2007). Among them, ultrasonography and blood marker testing are classified as screening tests, and amniocentesis is classified as a confirmation test. Ultrasonography and blood marker testing, which are noninvasive methods, are safe methods that do not comprise direct sampling from the fetus, but show a testing sensitivity of 80% or less (ACOG Committee on Practice Bulletins. 2007). Amniocentesis, chorionic villus sampling and percutaneous umbilical cord blood sampling, which are invasive methods, can confirm fetal chromosomal abnormalities, but have a disadvantage in that there is a possibility of losing the fetus due to invasive medical practices (Mujezinovic F, et al. 2007). In 1997, to et al. succeeded in Y chromosome sequencing of fetal-derived genetic material from maternal plasma and serum, and since then, fetal genetic materials in the maternal body have been used in prenatal testing (to Y M, et al. 1997). A fetal genetic material in maternal blood is produced when a portion of trophoblast cells which underwent the apoptotic process during placental remodeling enters the maternal blood through a material exchange mechanism. The fetal genetic material in fact originates from the placenta and is defined as off DNA (cell-free fetal DNA). cff DNA is found from 18 days after embryo transfer in fast cases and is found in most maternal blood on 37 days after embryo transfer (Guibert J, et al. 2003). cff DNA has characteristics in that it is a short strand having a length of 300 bp or less and is present in maternal blood in small amounts. Due to these characteristics, in order to apply cff DNA to the detection of fetal chromosomal abnormalities, massive parallel sequencing technology using the next-generation sequencer (NGS) has been used. Although noninvasive methods for detecting fetal chromosomal abnormalities using the massive parallel sequencing technology show a detection sensitivity of 90 to 99% or more depending on chromosomes, the false-positive and false-negative rates of the methods reach 1 to 10%, and thus a technology for correcting these false-positive and false-negative rates is urgently needed (Gil M M, et al. 2015).

Accordingly, the present inventors have made extensive efforts to solve the above-described problems and develop a method for detecting fetal chromosomal abnormalities with high sensitivity and low false-positive and false-negative rates, and as a result, have found that when fetal chromosomal regions are normalized and reference chromosomes are randomly permuted, analysis results with high sensitivity and low false-positive/false-negative rates can be obtained, thereby completing the present invention.

### DISCLOSURE OF INVENTION

### Technical Problem

It is an object of the present invention to provide a method for noninvasively detecting fetal gender and copy number abnormalities.

Another object of the present invention is to provide an apparatus for noninvasively detecting fetal gender and copy number abnormalities.

Still another object of the present invention is to provide a computer readable medium comprising instructions configured to be executed by a processor that detects fetal gender and copy number abnormalities by the above-described method.

### Technical Solution

To achieve the above object, the present invention provides a method for detecting fetal gender and copy number abnormalities, the method comprising the steps of:

a) obtaining reads from an extracted DNA from a maternal biological sample;

b) aligning the obtained reads to a reference genome database;

c) calculating Q-scores for the aligned reads, and selecting only reads which are equal to or lower than a cut-off value; and

d) calculating G-scores for the selected reads, and comparing the G-scores with those of a reference chromosome combination, thereby determining fetal gender and copy number variation.

The present invention also provides an apparatus for detecting fetal gender and copy number abnormalities, the apparatus comprising:

a) a reading unit for reading reads from an extracted DNA from a maternal biological sample and reading reads from the DNA;

b) an alignment unit for aligning the read reads to a reference genome database;

c) a quality control unit for calculating Q-scores for the aligned reads, and selecting only reads which are equal to or lower than a cut-off value; and

d) a gender and variation determining unit for calculating G-scores for the selected reads, and comparing the G-scores with those of a reference chromosome combination, thereby determining fetal gender and copy number variation.

The present invention also provides a computer readable medium comprising instructions configured to be executed by a processor that detects fetal gender and copy number abnormalities through the following steps: a) obtaining reads from an extracted DNA from a maternal biological sample; b) aligning the obtained reads to a reference genome database; c) calculating Q-scores for the aligned reads, and selecting only reads which are equal to or lower than a cut-off value; and d) calculating G-scores for the selected reads, and comparing the G-scores with those of a reference chromosome combination, thereby determining fetal gender and copy number variation.

### BRIEF DESCRIPTION OF THE DRAWINGS

FIG. 1 is an overall flow chart showing a method for detecting fetal gender and copy number abnormalities according to the present invention.

FIG. 2 depicts plots showing the correction results obtained before and after normalizing GC by the LOESS algorithm during quality control (QC) for read data.

FIG. 3 depicts plots showing the correction results Variation (CV) values by the LOESS algorithm during quality control (QC) for read data.

FIG. 4 depicts plots comparing G-score values calculated for a chromosomal abnormality group and a normal group according to a method of the present invention.

### BEST MODE FOR CARRYING OUT THE INVENTION

Unless defined otherwise, all the technical and scientific terms used herein have the same meaning as those generally understood by one of ordinary skill in the art to which the invention pertains. Generally, the nomenclature used herein and the experiment methods, which will be described below, are those well known and commonly employed in the art.

In the present invention, it has been found that when fetal gender and copy number abnormalities are detected by normalizing sequencing data obtained from a sample, aligning the normalized data based on a cut-off value, and then randomly permuting combinations of reference chromosomes to determine a reference chromosome combination in which the absolute value of a G-score difference between the chromosomes of a normal group and the chromosomes of a test subject satisfies the maximum value, analysis can be performed with high sensitivity and low false-positive/false-negative rates.

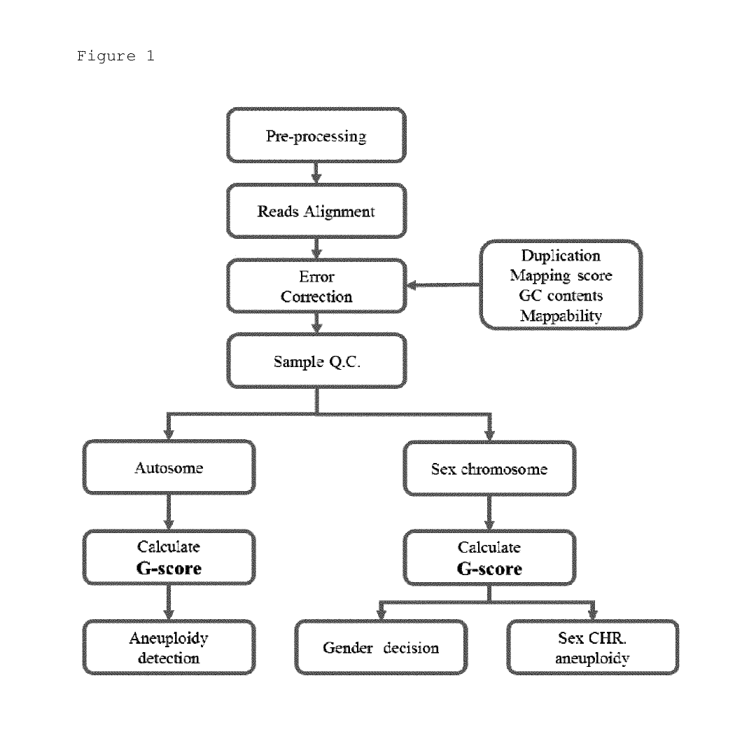

Namely, in an embodiment of the present invention, a method was developed which comprises: sequencing DNA extracted from maternal blood; controlling the quality of the sequence using the LOESS algorithm; calculating G-scores; randomly permuting reference chromosome combinations until the absolute value of a G-score difference between the chromosomes of a normal person group and the chromosomes of a test subject satisfies the maximum value; determining a cut-off value for the G-scores on the basis of the permutation results; and determining that there are abnormalities in the chromosomal copy number of the test subject, when the G-score of the test subject exceeds the cut-off value (FIG. 1).

Therefore, in one aspect, the present invention is directed to a method for detecting fetal gender and copy number abnormalities, the method comprising the steps of:

a) obtaining reads from an extracted DNA from a maternal biological sample;

b) aligning the obtained reads to a reference genome database;

c) calculating Q-scores for the aligned reads, and selecting only reads which are equal to or lower than a cut-off value; and

d) calculating G-scores for the selected reads, and comparing the G-scores with those of a reference chromosome combination, thereby determining fetal gender and copy number variation.

In the present invention, when the selected read is chromosome 13, the reference chromosome combination may be chromosomes 4 and 6, but is not limited thereto, when the selected read is chromosome 18, the reference chromosome combination may be chromosomes 4, 7, 10 and 16, but is not limited thereto, and when the selected read is chromosome 21, the reference chromosome combination may be chromosomes 7, 11, 14 and 22, but is not limited thereto. In addition, when the selected read is chromosome X, the reference chromosome combination may be chromosomes 16 and 20, but is not limited thereto, and when the selected read is chromosome Y, the reference chromosome combination may be chromosomes 1, 2, 3, 4, 5, 6, 7, 8, 9, 10, 11, 12, 14, 15, 17 and 19, but is not limited thereto.

In the present invention, step a) may comprise the steps of:

(i) obtaining a mixture of fetal and maternal nucleic acids from an amniotic fluid obtained by amniocentesis, a villus obtained by chorionic villi sampling, an umbilical cord blood obtained by percutaneous umbilical blood sampling, spontaneous miscarrying fetus tissue, or human peripheral blood;

(ii) removing protein, fat and other residues from the obtained mixture of fetal and maternal nucleic acids by a salting-out method, a column chromatography method or a bead-based method, and collecting purified nucleic acids;

(iii) constructing a single-end sequencing or pair-end sequencing library for the purified nucleic acids or nucleic acids randomly fragmented by an enzymatic cleavage, pulverization or hydroshear method;

(iv) subjecting the constructed library to a next-generation sequencer; and

(v) obtaining nucleic acid reads from the next-generation sequencer.

In the present invention, the next-generation sequencer may be a Hiseq system (Illumina Co.), a Miseq system (Illumina Co.), a Genome Analyzer (GA) system (Illumina Co.), 454 FLX sequencer (Roche Co.), a SOLiD™ system (Applied Biosystems Co.), or an Ion torrent system (Life Technology Co.), but is not limited thereto.

In the present invention, the alignment step may be performed using a BWA algorithm and a GRch38 sequence, but is not limited thereto.

In the present invention, step c) may comprise the steps of:

(i) specifying the region of each of the aligned nucleic acids sequence;

(ii) specifying a sequence that satisfies cut-off values for mapping quality score and CC content;

(iii) calculating the fraction of the chromosome (ChrN) of any case 1 in the specified sequence by use of the following equation 1:

$$\% ChrN = \frac{number of arranged sequences in chromosome N}{\begin{matrix}total number of aligned sequences \\ in chromosomes 1 to 22\end{matrix}} \qquad Equation 1$$

(iv) calculating Z-score for the chromosome N region by the following equation 2;

$$Z \text{-} score = \frac{\begin{matrix}( \% ChrN of any case 1 - \\ average \% ChrN of normal group )\end{matrix}}{standard deviation of \% ChrN of normal group} ↵ ; \qquad Equation 2$$

(v) calculating Q-score from the standard deviation of Z-scores for chromosomal regions other than regions corresponding to chromosomes 13, 18 and 21 of any case 1; and

(vi) determining a cut-off value for the Q-score, and determining that the Q-score is below standards, when the calculated Q-score exceeds the cut-off value, and reproducing reads from the sample of interest.

In the present invention, in step of (i) specifying the region of each of the aligned nucleic acids sequence, the region of each nucleic acid sequence may be 20 kb-1 MB, but is not limited thereto.

In the present invention, the mapping quality score in step (ii) may vary depending on the desired standard, but may preferably be 15-70 scores, more preferably 50-70 scores, most preferably 60 scores.

In the present invention, the GC content in step (ii) may vary depending on the desired standard, but may preferably be 20 to 70%, most preferably 30 to 60%.

In the present invention, the cut-off value in step (vi) may be 4, preferably 3, most preferably 2.

In the present invention, a case group means a sample for detecting fetal gender and chromosomal copy number abnormalities, and a reference group means a reference chromosomal group that is comparable such as a reference genome database, but is not limited thereto.

In the present invention, step of determining copy number variation in step d) may comprise the steps of:

(i) randomly selecting reference chromosomes from chromosomes 1 to 22;

(ii) calculating the fraction value of any chromosome N by the following equation 3:

$$\% G ( ChrN ) = \frac{number of arranged sequences in chromosome N}{\begin{matrix}number of randomly selected \\ reference chromosomes sequences\end{matrix}} \qquad Equation 3$$

(iii) calculating G-score for chromosome N of any case 1 by the following equation 4:

$$G \text{-} {score}_{case 1} ( ChrN ) = \frac{\begin{matrix}( \% G ( ChrN ) of case 1 - \\ average \% G ( ChrN ) of reference group )\end{matrix}}{\begin{matrix}standard deviation for G ( ChrN ) \% \\ value of reference group\end{matrix} } \qquad Equation 4$$

(iv) repeatedly performing steps (i) to (iii), thereby selecting a chromosome combination that maximizes the G-score difference between the normal group and the abnormal group; and

(v) calculating G-score using the chromosome combination obtained in step (iv), and determining that copy number decreased when the calculated G-score is lower than the cut-off value, and determining that copy number increased when the calculated G-score is higher than the cut-off value.

In the present invention, the number of the repeats in step (iv) may be 100 or more, preferably 1,000 or more, most preferably 100,000 or more.

In the present invention, the cut-off value for G-score in step (v) can be used without limitations as long as it is a value calculated for normal chromosomes, but may preferably be −2 or 2, most preferably −3 or 3, but is not limited thereto.

In the present invention, step of determining fetal gender in step d) may comprise the steps of:

(i) performing steps (i) to (iv) of determining copy number abnormalities in a maternal reference group in which the fetal karyotype is 46, XX or 46, XY, thereby obtaining G-score cut-off value for X and Y chromosomes; and

(ii) comparing G-scores for the X and Y chromosomes of any case with the cut-off values, thereby determining gender.

In the present invention, the G-score cut-off value for X and Y chromosomes may be −2 or 2, most preferably −3 or 3, but is not limited thereto. In the present invention, when the G-score for the X chromosome is lower than the cut-off value, it is determined that the sex chromosomes are XO, when the G-score for the X chromosome is higher than the cut-off value, it is determined that three or more X chromosomes are present, and when the G-score for the Y chromosome is higher than the cut-off value, it is determined that one or more Y chromosomes are present.

In the present invention, when one or more Y chromosomes are present, X chromosome fetal fraction may be calculated by the following equation 5 and Y chromosome fetal fraction may be calculated by the following equation 6 to thereby calculate the ratio of the Y chromosome fraction to the X chromosome fraction by the following equation 7, so that when the ratio is 0.7 to 1.4, it is determined that the sex chromosomes are XY, and when the ratio is 1.4 to 2.6, it is determined that the sex chromosomes are XYY:

$$\begin{matrix}X chromosome fetal fraction = 2 X ( 1 - \frac{\% chrX of any case 1}{\begin{matrix}\% chrX intermediate value of female \\ pregnant women control group\end{matrix}} ) & Equation 5 \\ Y chromosome fetal fraction = \frac{\begin{matrix}( \% chrY of any case 1 - \\ \% chrY of female pregnant women )\end{matrix}}{\begin{matrix}( \% chrY of male control group - \\ \% chrY of female pregnant women )\end{matrix}} & Equation 6 \\ Ratio of fraction = \begin{matrix}Y chromosome fetal fraction \\ X chromosome fetal fraction\end{matrix} . & Equation 7\end{matrix}$$

In another aspect, the present invention is directed to an apparatus for detecting fetal gender and copy number abnormalities, the apparatus comprising: a reading unit for extracting DNA from a maternal biological sample and reading reads from the DNA; an alignment unit for aligning the read reads to a reference genome database; a quality control unit for calculating Q-scores for the aligned reads, and selecting only reads which are equal to or lower than a cut-off value; and a gender and copy number variation determining unit for calculating G-scores for the selected reads, and comparing the G-scores with a reference chromosome combination, thereby determining fetal gender and copy number variation.

In the present invention, when the selected read is chromosome 13, the reference chromosome combination may be chromosomes 4 and 6, but is not limited thereto, when the selected read is chromosome 18, the reference chromosome combination may be chromosomes 4, 7, 10 and 16, but is not limited thereto, and when the selected read is chromosome 21, the reference chromosome combination may be chromosomes 7, 14 and 22, but is not limited thereto. In addition, when the selected read is chromosome X, the reference chromosome combination may be chromosomes 16 and 20, but is not limited thereto, and when the selected read is chromosome Y, the reference chromosome combination may be chromosomes 1, 2, 3, 4, 5, 6, 7, 8, 9, 10, 11, 12, 14, 15, 17 and 19, but is not limited thereto.

In the present invention, the reading unit may comprise: (i) a sampling unit for obtaining a mixture of fetal and maternal nucleic acids from an amniotic fluid obtained by amniocentesis, a villus obtained by chorionic villi sampling, an umbilical cord blood obtained by percutaneous umbilical blood sampling, spontaneous miscarrying fetus tissue, or human peripheral blood; (ii) a nucleic acid collecting unit for removing protein, fat and other residues from the obtained mixture of fetal and maternal nucleic acids by a salting-out method, a column chromatography method or a bead-based method, and collecting purified nucleic acids; (iii) a library constructing unit for constructing a single-end sequencing or pair-end sequencing library for the purified nucleic acids or nucleic acids randomly fragmented an enzymatic cleavage, pulverization or hydroshear method; (iv) a next-generating sequencing unit for subjecting the constructed library to a next-generation sequencer; and (v) a read acquiring unit for obtaining nucleic acid reads from the next-generation sequencer.

In the present invention, the next-generation sequencer may be a Hiseq system (Illumina Co.), a Miseq system (I-lumina Co.), a Genome Analyzer (GA) system (Illumina Co.), 454 FLX sequencer (Roche Co.), a SOLiD™ system (Applied Biosystems Co.), or an Ion torrent system (Life Technology Co.), but is not limited thereto.

In the present invention, the alignment unit may use a BWA algorithm and a GRch38 sequence, but is not limited thereto.

In the present invention, the quality control unit may comprise:

(i) a region specifying unit for specifying the region of each of the aligned nucleic acids sequence;

(ii) a sequence specifying unit for specifying a sequence that satisfies cut-off values for mapping quality score and GC content;

(iii) a chromosomal fraction calculating unit for calculating the fraction of the chromosome N (ChrN) of any case 1 in the specified sequence by use of the following equation 1:

$$\begin{matrix}\% ChrN = \frac{ \begin{matrix}number of arranged sequences \\ in chromosome N\end{matrix} }{\begin{matrix}total number of aligned sequences \\ in chromosomes 1 to 22\end{matrix}} & Equation 1 \\ Z - score = \frac{\begin{matrix}( \% ChrN of any case 1 - \\ average \% Chr N of normal group )\end{matrix}}{standard deviation of \% ChrN of normal group 1} ; & Equation 2\end{matrix}$$

(iv) a Q-score calculating unit for calculating Q-score from the standard deviation of Z-scores for chromosomal regions other than regions corresponding to chromosomes 13, 18 and 21 of any case 1; and

(v) a quality control unit for determining a cut-off value for the Q-score, and when the calculated Q-score exceeds the cut-off value, determining that the Q-score does not satisfy the cut-off, and reproducing reads from the sample of interest.

In the present invention, in the region specifying unit, the region of each nucleic acid sequence may be 20 kb-1 MB, but is not limited thereto.

In the present invention, the mapping quality score in the sequence specifying unit may vary depending on the desired standard, but may be preferably 13-70 scores, more preferably 50-70 scores, most preferably 60 scores.

In the present invention, the GC content in the sequence specifying unit may vary depending on the desired reference, but may preferably be 20 to 70%, most preferably 30 to 60%.

In the present invention, the cut-off value of the quality control unit may be 4, preferably 3, most preferably 2.

In the present invention, a case group means a sample for detecting fetal gender and chromosomal copy number abnormalities, and a reference group means a reference chromosomal group that is comparable such as a reference genome database, but is not limited thereto.

In the present invention, the copy number variation determination unit for determining copy number variation in the gender and copy number variation determining unit may comprise:

(i) a random permutation unit for randomly selecting reference chromosomes from chromosomes 1 to 22;

(ii) a chromosomal fraction calculating unit for calculating the fraction value of any chromosome N by the following equation 3:

$$\% G ( Chr N ) = \frac{\begin{matrix}number of arranged sequences \\ in chromosome N\end{matrix}}{\begin{matrix}number of randomly selected reference \\ chromosomes sequences\end{matrix}} \qquad Equation 3$$

(iii) a G-score calculating unit for calculating G-score for chromosome N of any case 1 by the following equation 4:

$$G \text{-} {score}_{case 1} ( ChrN ) = \frac{\begin{matrix}( \% G ( Chr N ) of case 1 - average \% G ( Chr N ) \\ of reference group )\end{matrix}}{\begin{matrix}standard deviation fo r \% G ( ChrN ) value of \\ reference group\end{matrix}} ; \qquad Equation 4$$

(iv) a reference chromosome combination selecting unit for repeatedly performing the operations of the units (i) to (iii), thereby selecting a chromosome combination that maximizes the G-score difference between the normal group and the abnormal group; and

(v) a copy number variation determining unit for calculating G-score using the chromosome combination selected in the reference chromosome combination selecting unit, and determining that copy number decreased when the calculated G-score is lower than the cut-off value, and determining that copy number increased when the calculated G-score is higher than the cut-off value.

In the present invention, the number of the repeats of the optimum reference chromosome combination G-score calculating unit may be 100 or more, preferably 1,000 or more, most preferably 100,000 or more.

In the present invention, the cut-off value for G-score of the copy number variation determining unit can be used without limitations as long as it is a value calculated for normal chromosomes, but may preferably be −2 or 2, most preferably −3 or 3, but is not limited thereto.

In the present invention, the gender determining unit in the fetal gender and copy number variation determining unit may comprise:

(i) a G-score cut-off calculating unit for performing the operations of the units (i) to (iv) of the copy number variation determining unit for determining copy number variation in a maternal reference group in which the fetal karyotype is 46, XX or 46, XY, thereby obtaining G-score cut-off value for X and Y chromosomes; and

(ii) a gender determining unit for comparing G-scores for the X and Y chromosomes of any case with the cut-off values, thereby determining gender.

In the present invention, the G-score cut-off value for X and Y chromosomes may be −2 or 2, most preferably −3 or 3, but is not limited thereto. In the present invention, when the G-score for the X chromosome is lower than the cut-off value, it is determined that the sex chromosomes are XO, when the G-score for the X chromosome is higher than the cut-off value, it is determined that three or more X chromosomes are present, and when the G-score for the Y chromosome is higher than the cut-off value, it is determined that one or more Y chromosomes are present.

In the present invention, when one or more Y chromosomes are present, X chromosome fetal fraction may be calculated by the following equation 5 and Y chromosome fetal fraction may be calculated by the following equation to thereby calculate the ratio of the Y chromosome fraction to the X chromosome fraction by the following equation 7, so that when the ratio is 0.7 to 1.4, it is determined that the sex chromosomes are XY, and when the ratio is 1.4 to 2.6, it is determined that the sex chromosomes are XYY:

$$\begin{matrix}X chromosome fetal fraction = 2 X ( 1 - \frac{\% chrX of any case 1}{\begin{matrix}\% chrX intermediate value of female \\ pregnant women control group\end{matrix}} ) & Equation 5 \\ Y chromosome fetal fraction = \frac{\begin{matrix}( \% chr Y of any case 1 - \% chrY of \\ female pregnant women )\end{matrix}}{\begin{matrix} ( \% chrY of male control group - \% chrY \\ of female pregnant women )\end{matrix}} , & Equation 6 \\ and & \\ Ratio of fraction = \frac{Y chromosome fetal fraction}{X chromosome fetal fraction} . & Equation 7\end{matrix}$$

In still another aspect, the present invention is directed to a computer readable medium comprising instructions configured to be executed by a processor that detects fetal gender and copy number abnormalities through the following steps: a) obtaining reads from an extracted DNA from a maternal biological sample; b) aligning the obtained reads to a reference genome database; c) calculating Q-scores for the aligned reads, and selecting only reads which are equal to or lower than a cut-off value; and d) calculating G-scores for the selected reads, and comparing the G-scores with a reference chromosome combination, thereby determining fetal gender and copy number variation.

### Examples

Hereinafter, the present invention will be described in further detail with reference to examples. It will be obvious to a person having ordinary skill in the art that these examples are for illustrative purposes only and are not to be construed to limit the scope of the present invention.

### Example 1: Next-Generation Sequencing of DNA Extracted from Maternal Blood

10 mL of maternal blood was sampled from each of a total of 358 pregnant women and stored in an EDTA tube. Within 2 hours after sampling, the blood was centrifuged at 1200 g at 4° C. for 15 minutes to obtain only plasma, and the plasma obtained by centrifugation was further centrifuged at 16000 g at 4° C. for 10 minutes to separate the plasma supernatant from the precipitant. From the separated plasma, cell-free DNA was extracted using a QIAamp Circulating Nucleic Acid Kit. 2 to 4 ng of the DNA was made into a library, and sequencing data were produced in a NextSeq system.

### Example 2: Quality Control of Sequencing Data

Sequencing data for the mixture of maternal-fetal genetic materials were pretreated, and a series of procedures were performed as follows before calculation of z-score. A Bcl file(including sequencing information) produced in a next-generation sequencer (NGS) system was converted into the fastq format, and then the library sequences in the fastq file were aligned to the reference genome Hg19 sequence by use of the BWA-mem algorithm. Since errors are likely to occur during alignment of the library sequences, three procedures for correcting errors were performed. First, an operation of removing overlapping library sequences was performed. Then, among the library sequences aligned by the BWA-mem algorithm, sequences that did not reach a mapping quality score of 60 were removed. Finally, areas with a mappability of 0.75 or less were removed, and a number of the library sequences aligned according to the chromosomal GC content were corrected using the LOESS algorithm. After a series of procedures as described above were performed, a bed file corrected for alignment errors was produced.

For the quality control of sequencing errors, a series of procedures were performed as follows. First, the relative fraction of each chromosome was calculated. For example, the relative fraction of chromosome 1 could be expressed as follows:

$$\% ChrN = \frac{number of arranged sequences in chromosome N}{\begin{matrix}total number of aligned sequences in \\ chromosomes 1 t o 22\end{matrix} } .$$

After the relative fractions of all chromosomes were calculated, Z-score for the chromosome-N region of case 1 could be expressed as follows:

$$Z - score = \frac{\begin{matrix}( \% Chr N of any case 1 - average \% ChrN \\ of normal group )\end{matrix}}{standard deviation of \% Chr N of normal group } .$$

The standard deviation of Z-scores for chromosomal regions other than regions corresponding to chromosomes 13, 18 and 21 could be expressed as Q-score.

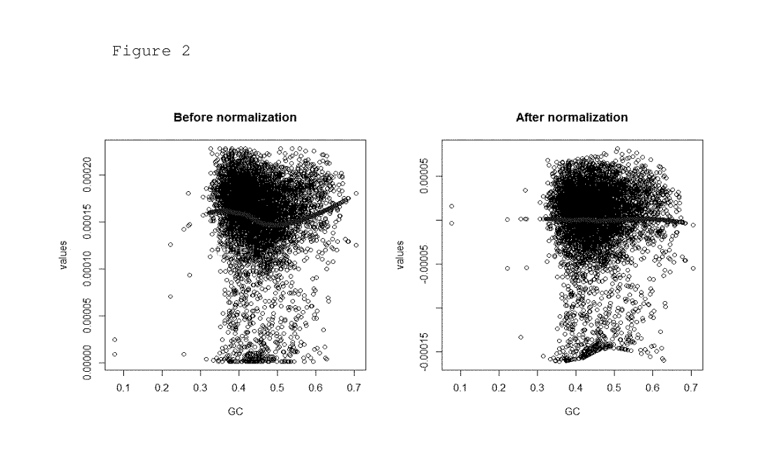

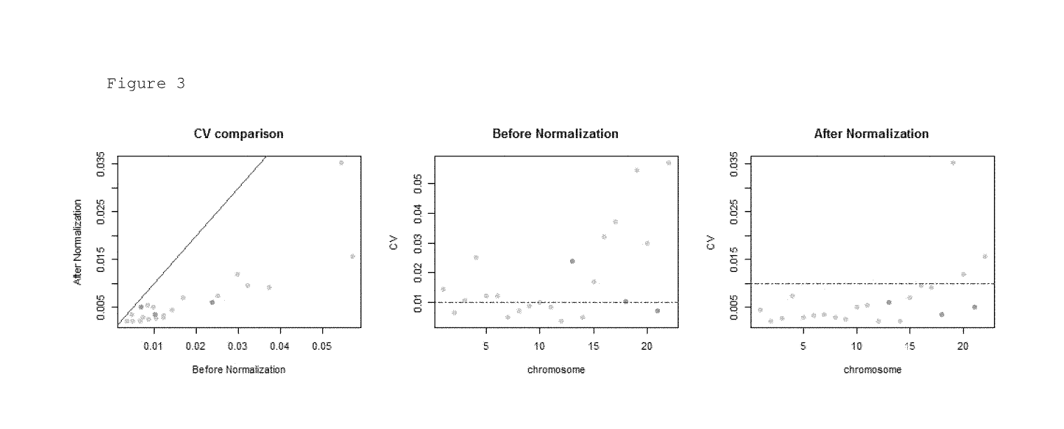

Thus, when the standard deviation value for the Z-score distribution of case 1 exceeded 2, it was determined to be QC-fail (sequencing error), and re-experimentation and data reproduction were performed. The above-described QC procedure was performed, and as a result, as can be seen in FIGS. 2 and 3, the distribution of reads was uniform.

### Example 3: G-Score Calculation and Determination of Fetal Gender/Copy Number Abnormalities Using Permutations

In order to calculate G-score, the following procedures were performed. First, the relative fraction of the chromosome of interest was calculated. For example, the relative fraction of a specific chromosome could be expressed as follows:

$$Relative fraction of chromosome N = \frac{number of aligned library sequences in chromosome N}{\begin{matrix}total number of aligned library sequences \\ in any chromosomes\end{matrix}}$$

The relative fraction of a specific chromosome may be expressed by the following equation 3:

$$\% G ( Chr N ) = \frac{\begin{matrix}number of arranged sequences \\ in chromosome N\end{matrix}}{\begin{matrix}number of randomly selected reference \\ chromosomes sequences\end{matrix}} \qquad Equation 3$$

In addition, for all chromosomes, the G-score of subject A could be expressed as follows:

$$G - score for chromosome N = \frac{\begin{matrix}relative fraction of chromosome N of subject A - \\ average relative fraction of chromosome N of normal persons\end{matrix}}{ \begin{matrix}standard deviation for relative fraction of chromosome N \\ of normal persons\end{matrix}}$$

The G-score may be expressed as the following equation 4:

$$G \text{-} {score}_{case 1} ( ChrN ) = \frac{\begin{matrix}( \% G ( Chr N ) of case 1 - average \% G ( Chr N ) \\ of reference group )\end{matrix}}{\begin{matrix}standard deviation fo r \% G ( ChrN ) value of \\ reference group\end{matrix}} \qquad Equation 4$$

The absolute value of the G-score difference between the chromosome N of the normal person group and the chromosome N of subject A was calculated, and random permutations were performed, thereby determining a reference chromosome combination in which the absolute value satisfied the maximum value. When the results were compared while random permutations increased, results with 50% or more improvement as shown in Table 1 below could be obtained by a large amount of permutation analyses.

TABLE 1
Results of random permutation analysis for
chromosomes 13, 18 and 21

| | Number of Random Permutations performed | | | | | | | | | |
|---|---|---|---|---|---|---|---|---|---|---|
| Chromosomes | 100 | 500 | 1,000 | 1,500 | 2,000 | 5,000 | 10,000 | 15,000 | 50,000 | 100,000 |
| 13 | 6.903 | 8.142 | 9.361 | 9.361 | 8.955 | 9.361 | 8.955 | 9.361 | 9.361 | 9.361 |
| 18 | −0.52 | −0.09 | −0.025 | −0.012 | −0.025 | −0.025 | 0.122 | 0.136 | 0.128 | 0.136 |
| 21 | 1.051 | 1.352 | 1.343 | 1.364 | 1.168 | 1.201 | 1.352 | 1.374 | 1.377 | 1.532 |

The reference chromosome combination could be changed by an optimization operation in each analysis, and combinations detected in 5 or more of 10 operations performed to determine the G-scores of chromosomes 13, 18, 21, X and Y could be obtained as shown in Table 2 below.

TABLE 2
Major reference chromosome combinations
used to calculate the G-scores of chromosomes 13, 18, 21,
X and Y

| Chromosomes | |
|---|---|
| of interest | Reference Chromosome Combination |
| 13 | 4 and 6 |
| 18 | 4, 7, 10 and 16 |
| 21 | 7, 11, 14 and 22 |
| X | 16 and 20 |
| Y | 1, 2, 3, 4, 5, 6, 7, 8, 9, 10, 11, 12, 14, |
| | 15, 17 and 19 |

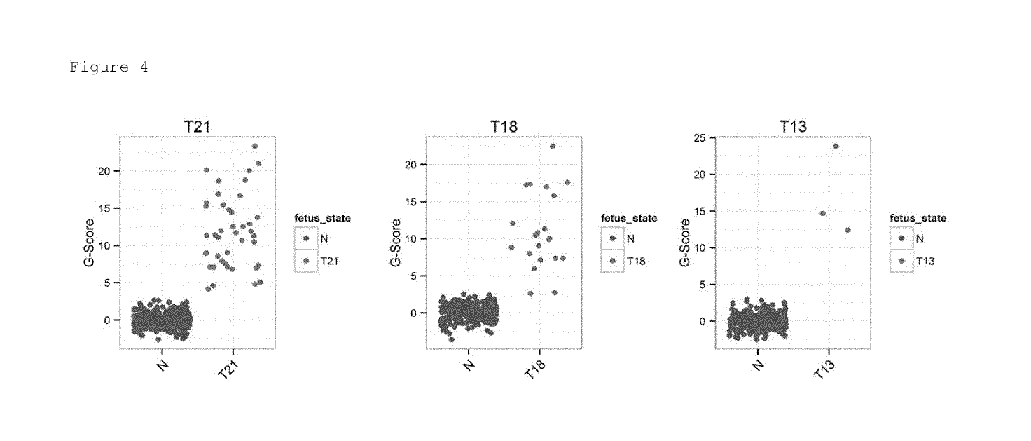

In order to determine whether the chromosome of interest in a test sample would be aneuploidy, the G-score range of a normal group was calculated and established. When a outlier deviating from the greatest and smallest G-scores of the normal group was found, it was determined that chromosomal aneuploidy was detected. When the outlier was greater than the greatest G-score of the normal group, it was determined that the copy number of the chromosome of interest was added, and when the outlier was smaller than the smallest G-score of the normal group, the copy number of the chromosome of interest was lost. Chromosomal abnormality groups (Trisomy 21, Trisomy 18, and Trisomy 13) were compared with the normal group by the above-described method, and as a result, it could be seen that the greatest and smallest G-scores were not consistent between the chromosomal abnormality groups and the normal group (FIG. 4). In addition, as can be in Table 3 below, when the G-score cut-off values for chromosomal aneuploidy were 3 (Trisomy 21), 2.55 (Trisomy 18) and 3.5 (Trisomy 13), respectively, chromosomal abnormalities (increased copy numbers) were detected with a sensitivity of 100% and a specificity of 100%, and the lower limit of the 95% confidence interval of specificity was higher than 98%.

TABLE 3
Sensitivity and specificity of chromosomal
abnormality detection by G-score calculation method

| | Sensitivity (95% | Specificity (95% |
|---|---|---|
| Chromosomal | confidence | confidence |
| abnormalites | interval) | interval) |
| Trisomy 21 | 100% (91.62-100.0%) | 100% (98.80-100.0%) |
| (n = 42) | | |
| Trisomy 18 | 100% (84.54-100.0%) | 100% (98.87-100.0%) |
| (n = 21) | | |
| Trisomy 13 | 100% (43.85-100.0%) | 100% (98.93-100.0%) |
| (n = 3) | | |

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described above, the method for determining fetal gender and chromosomal copy number abnormalities according to the present invention can detect fetal gender with more increased accuracy by next-generation sequencing (NGS), and can also detect sex chromosomal abnormalities such as XO, XXX, XXY and the like, which have been difficult to detect, with more increased accuracy, so that the commercial utilization of the method can be increased. Accordingly, the method of the present invention can be effectively used for prenatal diagnosis to early detect malformation caused by fetal sex chromosomal abnormalities.

1. A method for detecting fetal gender and copy number abnormalities, the method comprising:
a) obtaining reads from an extracted DNA from a maternal biological sample;
b) aligning the obtained reads to a reference genome database;
c) calculating Q-scores for the aligned reads, and selecting only reads which are equal to or lower than a cut-off value; and
d) calculating G-scores for the selected reads, and comparing the G-scores with those of a reference chromosome combination, thereby determining fetal gender and copy number variation.

2. The method of claim 1, wherein the reference chromosome combination in step d) is chromosomes 4 and 6 when the selected read is chromosome 13, the reference chromosome combination is chromosomes 4, 7, 10 and 16 when the selected read is chromosome 18, the reference chromosome combination is chromosomes 7, 11, 14 and 22 when the selected read is chromosome 21, the reference chromosome combination is chromosomes 16 and 20 when the selected read is chromosome X, and the reference chromosome combination is chromosomes 1, 2, 3, 4, 5, 6, 7, 8, 9, 10, 11, 12, 14, 15, 17 and 19, when the selected read is chromosome Y.

3. The method of claim 1, wherein step a) comprises the steps of:
(i) obtaining a mixture of fetal and maternal nucleic acids from an amniotic fluid obtained by amniocentesis, a villus obtained by chorionic villi sampling, an umbilical cord blood obtained by percutaneous umbilical blood sampling, spontaneous miscarrying fetus tissue, or human peripheral blood;
(ii) removing protein, fat and other residues from the obtained mixture of fetal and maternal nucleic acids by a salting-out method, a column chromatography method or a bead-based method, and collecting purified nucleic acids;
(iii) constructing a single-end sequencing or pair-end sequencing library for the purified nucleic acids or nucleic acids randomly fragmented by an enzymatic cleavage, pulverization or hydroshear method;
(iv) subjecting the constructed library to a next-generation sequencer; and
(v) obtaining nucleic acid reads from the next-generation sequencer.

4. The method of claim 1, wherein step c) comprises the steps of:
(i) specifying the region of each of the aligned nucleic acids sequence;
(ii) specifying a sequence that satisfies cut-off values for mapping quality score and GC content;
(iii) calculating the fraction of the chromosome N (ChrN) of any case 1 in the specified sequence by use of the following equation 1:
$$\% ChrN = \frac{\begin{matrix}number of arranged sequences \\ in chromosome N\end{matrix}}{\begin{matrix}total number of aligned sequences \\ in chromosomes 1 to 22\end{matrix}} \qquad Equation 1$$
(iv) calculating Z-score for the chromosome N region by the following equation 2;
$$Z - score = \frac{\begin{matrix}( \% ChrN of any case 1 - \\ average \% Chr N of normal group )\end{matrix}}{standard deviation of \% ChrN of normal group} ; \qquad Equation 2$$
(v) calculating Q-score from the standard deviation of Z-scores for chromosomal regions other than regions corresponding to chromosomes 13, 18 and 21 of any case 1; and
(vi) determining a cut-off value for the Q-score, and -determining that the Q-score is below standards, when the calculated Q-score exceeds the cut-off value, and reproducing reads from the sample of interest.

5. The method of claim 4, wherein the mapping quality score in step (ii) is 15-70 scores, and the GC content satisfies 30 to 60%.

6. The method of claim 4, wherein the cut-off value in step (vi) is 4.

7. The method of claim 1, wherein step d) comprises the steps of:
(i) randomly selecting reference chromosomes from chromosomes 1 to 22;
(ii) calculating the fraction value of any chromosome N by the following equation 3:
$$\% G ( Chr N ) = \frac{\begin{matrix}number of arranged sequences \\ in chromosome N\end{matrix}}{\begin{matrix}number of randomly selected reference \\ chromosomes sequences\end{matrix}} \qquad Equation 3$$
(iii) calculating G-score for chromosome N of any case 1 by the following equation 4:
$$G \text{-} {score}_{case 1} ( ChrN ) = \frac{\begin{matrix}( \% G ( Chr N ) of case 1 - average \% G ( Chr N ) \\ of reference group )\end{matrix}}{\begin{matrix}standard deviation fo r \% G ( ChrN ) value of \\ reference group\end{matrix}} ; \qquad Equation 4$$
(iv) repeatedly performing steps (i) to (iii), thereby selecting a chromosome combination that maximizes the G-score difference between the normal group and the abnormal group; and
(v) calculating G-score using the chromosome combination obtained in step (iv), and determining that copy number decreased when the calculated G-score is lower than the cut-off value, and determining that copy number increased when the calculated G-score is higher than the cut-off value.

8. The method of claim 1, wherein step of determining fetal gender in step d) comprises the steps of:
(i) performing steps (i) to (iv) of claim 7 in a maternal reference group in which the fetal karyotype is 46, XX or 46, XY, thereby obtaining G-score cut-off value for X and Y chromosomes; and
(ii) comparing G-scores for the X and Y chromosomes of any case with the cut-off values, thereby determining gender.

9. The method of claim 8, wherein when the G-score for the X chromosome is lower than the cut-off value, it is determined that the sex chromosomes are XO, wherein when the G-score for the X chromosome is higher than the cut-off value, it is determined that three or more X chromosomes are present, and wherein when the G-score for the Y chromosome is higher than the cut-off value, it is determined that one or more Y chromosomes are present.

10. The method of claim 9, wherein when one or more Y chromosomes are present, X chromosome fetal fraction is calculated by the following equation 5 and Y chromosome fetal fraction is calculated by the following equation 6 to thereby calculate the ratio of the Y chromosome fraction to the X chromosome fraction by the following equation 7, so that when the ratio is 0.7 to 1.4, it is determined that the sex chromosomes are XY, and when the ratio is 1.4 to 2.6, it is determined that the sex chromosomes are XYY:
$$\begin{matrix}X chromosome fetal fraction = 2 X ( 1 - \frac{\% chrX of any case 1}{\begin{matrix}\% chrX intermediate value of female \\ pregnant women control group\end{matrix}} ) & Equation 5 \\ Y chromosome fetal fraction = \frac{\begin{matrix}( \% chr Y of any case 1 - \% chrY of \\ female pregnant women )\end{matrix}}{\begin{matrix} ( \% chrY of male control group - \% chrY \\ of female pregnant women )\end{matrix}} , & Equation 6 \\ and & \\ Ratio of fraction = \frac{Y chromosome fetal fraction}{X chromosome fetal fraction} . & Equation 7\end{matrix}$$

11. The method of claim 7, wherein the cut-off value is −2 or 2.

12. The method of claim 7, wherein the number of the repeats in step (iv) is 100 or more.

13. An apparatus for detecting fetal gender and copy number abnormalities, the apparatus comprising:
a reading unit for reading reads from an extracted DNA from a maternal biological sample and reading reads from the DNA;
an alignment unit for aligning the read reads to a reference genome database;
a quality control unit for calculating Q-scores for the aligned reads, and selecting only reads of the sample which are equal to or lower than a cut-off value; and
a gender and copy number variation determining unit for calculating G-scores for the selected reads, and comparing the G-scores with those of a reference chromosome combination, thereby determining fetal gender and copy number variation.

14. A computer readable medium comprising instructions configured to be executed by a processor that detects fetal gender and copy number abnormalities through the following steps:
a) obtaining reads from an extracted DNA from a maternal biological sample;
b) aligning the obtained reads to a reference genome database;
c) calculating Q-scores for the aligned reads, and selecting only reads which are equal to or lower than a cut-off value; and
d) calculating G-scores for the selected reads, and comparing the G-scores with those of a reference chromosome combination, thereby determining fetal gender and copy number variation.
